(19) United States
(12) Patent Application Publication
(10) Pub. No.: US 2016/0007914 A1
(43) Pub. Date: Jan. 14, 2016

(54) SLEEP CONTROL DEVICE

(71) Applicants: Jason Xu, San Jose, CA (US); Hongpeng Wang, Sunnyvale, CA (US); Heng Cao, San Jose, CA (US); David Peng Xu, San Jose, CA (US); Cien Shen, Sunnyvale, CA (US)

(72) Inventors: Jason Xu, San Jose, CA (US); Hongpeng Wang, Sunnyvale, CA (US); Heng Cao, San Jose, CA (US); David Peng Xu, San Jose, CA (US); Cien Shen, Sunnyvale, CA (US)

(21) Appl. No.: 14/796,996

(22) Filed: Jul. 10, 2015

Related U.S. Application Data
(60) Provisional application No. 62/024,363, filed on Jul. 14, 2014.

(51) Int. Cl. A61B 5/00; A61B 5/0476; A61B 5/0402; A61B 5/145; A61B 5/11; A61B 3/113

(52) U.S. Cl. CPC A61B 5/486; A61B 5/4815; A61B 5/11; A61B 5/0053; A61B 5/0057; A61B 5/0051; A61B 5/6801; A61B 5/7271; A61B 5/4818; A61B 3/113; A61B 5/0402; A61B 5/14542; A61B 5/0476; A61B 5/7455

(57) ABSTRACT

Systems, apparatus, and methods of monitoring and reducing snore are discussed herein. Some embodiments may provide for a system including a snore detection module, a movement detection module, a control module, and an actuation module. The snore detection module may be configured to detect snore, such as by detecting vibrations caused by snoring. When snoring is detected, the control module may be configured to instruct the actuation module to apply stimulation to the user that is calibrated to cause the user to shift sleeping position without disturbing sleep. The movement detection module may be configured to monitor user movement. If the user fails to move in response to the actuation, the actuation module may increase the intensity of the actuation. If the user responds to the actuation, the process may be repeated after a predetermined delay to provide continuous snore monitoring and correction throughout user sleep.

### FIELD

This application claims the benefit of U.S. Provisional Patent Application No. 62/024,363, titled “Snore Control Device,” filed Jul. 14, 2014, which is incorporated by reference herein in its entirety.

### TECHNICAL FIELD

The disclosure relates to sleep control, and more specifically, relates to the detection and reduction of snoring or sleep apnea.

### BACKGROUND

Snoring is common among different age groups. As people age, extra tissue may form around the nose, tongue, and throat of the upper respiratory airway, and associated muscles may lose tension. These factors, among others, may cause an individual to snore, which results in lower quality sleep for the individual and the individual's bed partner. People with various chronic diseases, such as diabetes or high blood pressure, may be at a greater risk when they have poor quality of sleep. Furthermore, chronic snoring that is untreated may progress to more severe symptoms, such as sleep apnea which may be life threatening.

### BRIEF SUMMARY

Systems, apparatuses, methods, and computer readable program code are provided to, in general, improve detection and reduction of snoring and/or sleep apnea. For example, some embodiments may include A sleep control system, including a snore detection module, a control module, and an actuation module. The snore detection module may be configured to: detect vibrations caused by snoring of a user; and generate vibration signals indicating the vibrations. The control module may be configured to: determine a vibration strength based upon the vibration signals; determine a vibration strength threshold; and determine whether the vibration strength exceeds the vibration strength threshold; and in response to determining that vibration strength exceeds the vibration strength threshold, provide an actuation signal to an actuation module. The actuation module may be configured to generate an actuation to stimulate movement of the user in response to receiving the actuation signal.

Some embodiments may include a machine-implemented method. The method may include: detecting, by a snore detection module, vibrations caused by snoring of a user; generating, by the snore detection module, vibration signals indicating the vibrations; determining, by a control module, a vibration strength based upon the vibration signals; determining, by the control module, a vibration strength threshold; determining, by the control module, whether the vibration strength exceeds the vibration strength threshold; in response to determining that vibration strength exceeds the vibration strength threshold, providing, by the control module, an actuation signal to an actuation module; and generating, by an actuation module, an actuation to stimulate movement of the user in response to receiving the actuation signal.

Some embodiments may include a device or apparatus, including circuitry configured to: receive vibration signals from a snore detection module indicating vibrations caused by snoring; determine a vibration strength based upon the vibration signals; determine a vibration strength threshold; and determine a vibration pattern based on the vibration signals determine a reference vibration pattern representative of snoring of the user; determine whether the vibration strength exceeds the vibration strength threshold and whether the vibration pattern corresponds with the reference vibration pattern; and in response to determining that vibration strength exceeds the vibration strength threshold and the vibration pattern corresponds with the reference vibration pattern, provide an actuation signal to an actuation module

Some embodiments may include a device, such as a wearable device, including: a fabric material; and a snore detection module attached with the fabric material, including: a vibration detector including an ionic polymer metal composite (IPMC), the vibration detector configured to detect vibrations caused by snoring of a user; and processing circuitry configured to: generate vibration signals indicating the vibrations; and provide the vibration signals to a control module.

Some embodiments may include one or more machines, such as an apparatus and/or system, configured to implement the methods and/or other functionality discussed herein. For example, the machine may include one or more processors and/or other machine components configured to implement the functionality discussed herein based on instructions and/or other data stored in memory and/or other non-transitory computer readable media.

These characteristics as well as additional features, functions, and details of the present invention are described below. Similarly, corresponding and additional embodiments are also described below.

### BRIEF DESCRIPTION OF THE DRAWINGS

Having thus described some embodiments in general terms, reference will now be made to the accompanying drawings, which are not necessarily drawn to scale. The embodiments illustrated in the figures of the accompanying drawings herein are by way of example and not by way of limitation, and wherein:

FIG. 1 illustrates a block diagram of sleep control device in accordance with exemplary embodiments of the disclosure;

FIG. 2 illustrates a flow chart of a method for controlling snore in accordance with exemplary embodiment of the disclosure; and

FIG. 3 illustrates a schematic diagram of example circuitry in accordance with exemplary embodiments of the disclosure.

### DETAILED DESCRIPTION OF THE EMBODIMENTS

The subject disclosure now will be described more fully hereinafter with reference to the accompanying drawings, in which preferred embodiments are shown. This disclosure may, however, be embodied in many different forms and should not be construed as limited to the embodiments set forth herein; rather, these embodiments are provided so that this disclosure will be thorough and complete, and will fully convey the scope of the disclosure to those skilled in the art. In this regard, reference may be made herein to a number of mathematical or numerical expressions or values, and to a number of positions of various components, elements or the like. It should be understood, however, that these expressions, values, positions or the like may refer to absolute or approximate expressions, values or positions, such that exemplary embodiments may account for variations that may occur, such as those due to engineering tolerances. Like numbers refer to like elements throughout.

As used herein, the word “exemplary” is used herein to refer to serving as an example, instance, or illustration. Any aspect, feature, function, design, etc. described herein as “exemplary” or an “example” or “examples” is not necessarily to be construed as preferred or advantageous over other aspects, features, functions, designs, etc. Rather, use of the word exemplary is intended to present concepts in a concrete fashion.

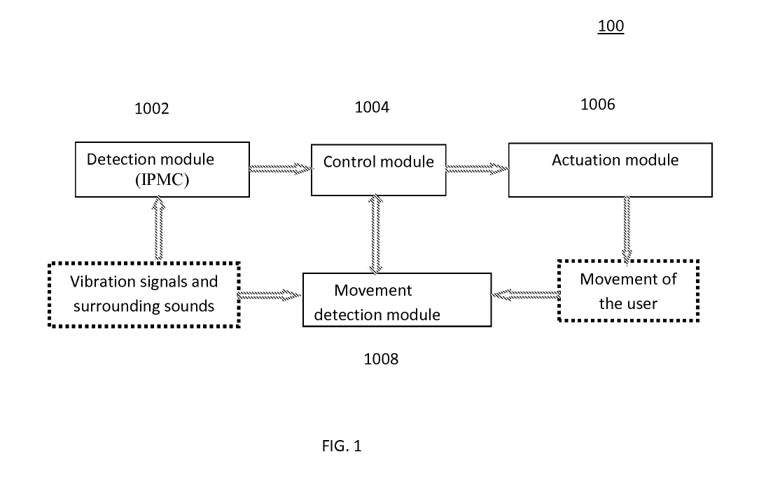

FIG. 1 illustrates a block diagram of sleep control system 100 in accordance with exemplary embodiments of the disclosure. Sleep control system 100 may comprise a snore detection module 1002, a control module 1004, an actuation module 1006 and a movement detection module 1008.

Snore detection module 1002 may be configured to detect the snoring of a user. For example, snore detection module 1002 may include a vibration detector that is configured to detect vibrations caused by the user's snoring, such as from sound transmitted through the air, and/or vibrations through the user's body and any intervening objects capable of carrying the vibrations from the body to the vibration detector. The vibration detector may be disposed at or near the user's neck or chin areas. In some embodiments, snore detection module 1002 and/or the vibration detector may be attached with a fabric material, a collar, tie, necklace, bedding, or other object capable of securing the vibration detector near the user's neck or chin.

In some embodiments, the vibration detector of snore detection module 1002 may include an ionic polymer metal composite (IPMC) used as a micro-vibration detector to detect the vibration signals. IPMC is an electro-active polymer material comprising an ion exchange membrane with electro-less plating metal material (e.g., on both sides of the ion exchange membrane), such as nickel, copper, silver, gold, platinum, palladium, and/or the like. The IPMC may be a thin (e.g., 0.2˜1 mm thick) electro-active material with high flexibility.

When using IPMC within the snore detection module 1002 and worn by a single user, the IPMC is capable of detecting vibrations without picking up surrounding sounds (e.g., as may be picked up by a microphone). Furthermore, another advantage of IPMC over a microphone detector is that the strength and quality of the vibrational signal does not depend on the orientation of the snoring user or the relative positions of the microphone and the user's mouth. For example, a microphone would be less able to detect snore when the sound source is facing away or a greater distance from the microphone. The microphone may also pick up surrounding sound, such as snoring sound of a bed partner or surrounding noises. Picking up unwanted sound signals may affect accuracy of the monitoring. In some embodiments, however, snore detection module 1002 may additionally or alternatively include a microphone for detecting the snoring of the user. In some embodiments, snore detection module 1002 may alternatively or additionally include a transducer and/or a piezoelectric material.

When user starts to snore, the snore detection module 1002 may detect the vibration (e.g., sound), generate vibration signals in response to detecting the vibration, and transmit the vibration signals to control module 1004. In some embodiments, the snore detection module may further include a wireless transmitter configured to wirelessly transmit the vibration signals.

Control module 1004 may be configured to receive the vibration signals and process the vibration signals. Control module 1004 may determine vibration strength of the vibration signals and compare the vibration strength to one or more predetermined vibration strength thresholds. When control module 1004 determines vibration strength is stronger than a predetermined vibration threshold (and/or the signal pattern matches a reference pattern), control module 1004 may generate a control signal, such as an actuation signal (e.g., including an actuation code), and transmit the control signal to the actuation module 1006. When snore detection module 1002 picks up surrounding audio signals (e.g., via a microphone), the surrounding audio signals may also be transmitted to control module 1004. Detection module 1002 may be configured to use voice recognition and/or pattern recognition of IPMC vibrations to distinguish snoring sounds from surrounding noises, thus identifying useful data for analysis and processing. In some embodiments, control module 1004 may be implemented by an electronic device (e.g., a smart phone, media player, tablet, laptop, desktop, etc.) on which an application may be installed.

Actuation module 1006 may be configured to generate an actuation to cause the user to change his/her sleep position. Studies indicate that individuals who sleep in the supine position (on the back) may be more likely to snore than those who sleep in the lateral position (on the side). Thus the actuation may cause user who is originally sleeping in the supine position to shift to the lateral position, thereby reducing the snoring.

Actuation module 1006 may include a vibration generator, such as an electronic motor (e.g., with or without linear motion capabilities). As such, actuation module 1006 may be disposed at or near the user's body to apply the actuation to the user as a vibrational stimulation. The vibration generator may be in various forms, such as a button which can be attached with a fabric material on the user's clothing (e.g., pajamas), inserted into or under a pillow, attached to a wristband, watch, neckband, or headband, attached as part of a bedding sheet or mattress among other things. In some embodiments, the vibration generator may include an IPMC configured to generate the vibrational stimulus, such as in response an actuation signal. In some embodiments, the actuation module may further include a wireless receiver configured to wirelessly receive the actuation signals.

In some embodiments, actuation module 1006 may be configured to apply one or more different actuation types, such as one or more of a vibration, a pressure, a massage, an air pressure (hot or cold blown air), a menthol preparation and electrical stimulation (e.g., touch), and/or a stimulation of vision, smell, sound (e.g., music), and taste. In some embodiments, an actuation may further include a communication to a designated individual, such as via telephone call, email, text message, application alert, etc. Actuation module 1006 may include one or more of a swipe actuator, a heat/cold transmission unit, a pressure generator and/or an electronic vibration generator. For example, actuation module 1006 may apply heat that is warmer than the user's body temperature or apply cold that is colder than user's body temperature, thereby stimulating user's skin. By stimulating user's body or skin, the user may change his/her sleep position.

In some embodiments, the actuation or stimulation strength may be adjusted based on the placement of actuation module 1006. For example, when actuation module 1006 is inserted into the pillow, the stimulation strength may be lower than when actuation module 1006 is worn on the user's body. The stimulation strength may also be adjusted and customized for each individual user. For example, some users may be more sensitive to cold stimulation while other users may be more sensitive to heat stimulation. Therefore, the actuation may be determined and adjusted in accordance with each user's circumstances. The stimulation strength may be measured by frequency, amplitude, voltage, ampere, Celsius or pascal. The stimulation strength may additionally or alternatively be measured by a style of the stimulation, such as different lengths of actuation and stoppage, and/or different rates of intensity increase such as flat, linear, and step increases. The actuation signal may be linear or nonlinear depending on different applications. For example, the frequency and/or amplitude of the actuation signal may be adjusted in a linear or nonlinear way based on different applications or configurations of the actuation module 1006.

If the stimulation applied to the user makes the user change move or position, movement/position detection module 1008 may detect the movement and generate a motion signal in response to the movement. In absence of a movement of the user, the motion signal may indicate an absence of motion, which may also be transmitted to control module 1004. Control module 1004 may determine, based on the absence of motion, that the actuation may be too slight for the user and send a control signal to actuation module 1006 to increase the intensity of the actuation. The control signal may cause actuation module 1006 to increase the stimulation intensity which may generate a stronger stimulation. With the stronger stimulation, the user may be caused to move or change sleep position. In some embodiments, control module 1004 may be configured to continue increase the actuation intensity (e.g., up to a preset maximum) until the user is determined to have moved and/or decreased snoring. Movement detection module 1008 may comprise one or more sensors (e.g., accelerometers, gyroscopes, etc.) to detect movement signals caused by movement of the body. In some embodiments, movement detection module 1008 may further include a wireless transmitter configured to wirelessly transmit the movement signals.

In some embodiments, system 100 may include one or more sleep sensors for monitoring the quality of sleep, such as electrocardiogram (ECG) and/or blood oxygen sensors. The one or more sleep sensors may be included with at least one of snore detection module 1002, actuation module 1006, and/or movement detection module 1008. In another example, system 100 may include a sleep sensor module including the one or more sleep sensors. The sleep sensors may include a motion sensor, body position sensor, eye movement sensor, heart rate sensor, blood pressure sensor, temperature sensor, brain activity sensor, bed quality sensor, sleeping environment sensor, ECG sensor, blood oxygen sensor, other health-related sensor, etc. The sleep sensor module may be configured to provide sleep sensor data to control module 1004, which may be processed by control module 1004 or a central system.

In some embodiment, control module 1004 collect data from snore detection module 1002, movement detection module 1008, and/or other sensor, and transmit the data to a central system, server, or data processing center, such as a cloud center. The central system may analyze the data and generate a preferred actuation type and/or intensity based on the analysis. For example, the preferred actuation may be determined based on an individual user's data and/or data collected across multiple users. In some embodiments, the central system may be further configured to monitor or assist user sleep, such as by determining based on received data, a sleep quality index, sleep improvement techniques, risk or symptoms of sleep-related illness (e.g., sleep apnea), bed suitability, etc. The central system may further provide information to control module 1004 to provide the determined data as recommendations to control module 1004 to improve the user's sleep.

In some embodiments, control module 1004 and/or the central system may be configured to determine and monitor sleep apnea. For example, system 100 may include sensors suitable for sleep apnea detection such as the vibration detector, the microphone detector, an eye movement sensor, ECG sensor, blood oxygen sensor, and/or a brain wave sensor. Control module 1004 and/or the central system may be further configured to determine whether the user is in a state of sleep apnea based on the sensor data from the sensors, such as by comparing the signals received from the sensors with reference patterns, indicators, and/or threshold strengths that are characteristic of sleep apnea.

In some embodiments, the control module 1004 and/or the central system may be configured to determine sleep apnea based on the vibration signals generated by the snore detection module. For example, the control module 1004 and/or the central system may be configured to compare the vibration signals received from the snore detection module with reference patterns, indicators, and/or threshold strengths that are characteristic of sleep apnea. An example reference pattern for sleep apnea may include detected snoring that is interrupted for a predefined amount of time. In some embodiments, the predefined amount of time (e.g., 2 seconds), or other pattern characteristics, may be determined based on crowd sourced user data that is aggregated from multiple users. In some embodiments, sleep apnea may be determined based on a combination of the vibratory signals and sensor data.

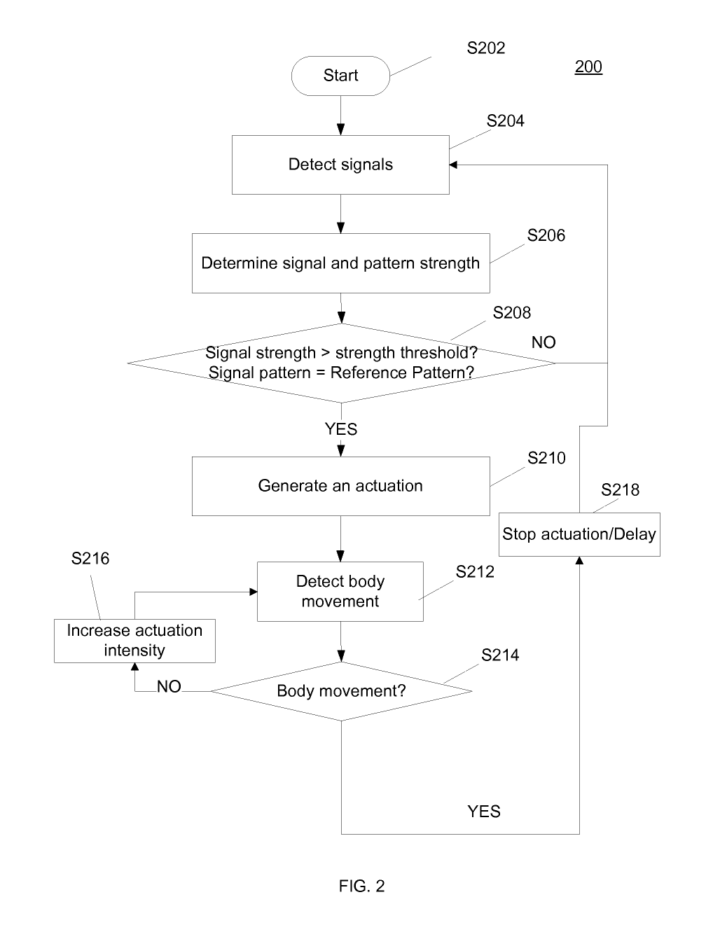

In some embodiments, control module 1004 may be configured to perform some or all of the features discussed herein with respect to the central system. Similarly, the central system may be configured to perform one or more of the steps discussed herein with respect to control module 1002 (e.g., as shown in method 200 and FIG. 2).

In various embodiments, a single device of system 100 may include one or more of modules 1002-1008 that share a common housing or are otherwise mechanically attached with each other. In that sense, modules 1002-1008 may be distributed across one or more separate devices, may be separate parts, or combined into any shape, form and combination as suitable. For example, in some embodiments, snore detection module 1002, actuation module 1006, and/or movement detection module 1008 may be part of a wearable device while control module 1004 may be part of a separate processing device, such as a mobile phone, smart phone, tablet, desktop, laptop, and server, among other things. Here, the modules of the wearable device may share common hardware, such as processing circuitry and/or a wireless transmitter. Where two modules discussed herein are as communicating with each other are included with separate devices, the two modules may communicate based on wired and/or wireless communications, and may include suitable transmitters, receivers, ports, etc.

In another example, one or more of snore detection module 1002, actuation module 1006, and movement detection module 1008 may be separate from each other, such that various modules can be located at or near different locations of the user's body.

In some embodiments, movement detection module 1008, snore detection module 1002, actuation module 1006, and/or control module 1004 may be attached to a common fabric material or other article.

FIG. 2 illustrates a flow chart of a method 200 for controlling snore in accordance with exemplary embodiments. Method 200 may start at S202 and proceed to S204, where a snore detection module, such as snore detection module 1002 as illustrated in FIG. 1, may be configured to detect vibrations and generate vibration signals. The vibration signals include vibrations detected by snore detection module 1002 through objects, such as vibrations that originate from the user's body, through one or more objects (e.g., a portion of a wearable collar, neckband, etc.) or otherwise, and to snore detection module 1002. In some embodiments, the snore detection module may be further configured to detect sound and generate audio signals. The audio signals, when used, may include the snoring sound or surrounding environmental noise traveling from the sound source, through the air, to snore detection module 1002.

At S206, a control module, such as control module 1004, may be configured to receive and process the generated vibration signals (and/or audio signals) and determine signal strength and signal pattern. The signal strength may indicate an intensity of the detected vibration, and the signal pattern may indicate the vibrational frequency, changes to intensity over time, among other things.

At S208, the control module may be configured to determine whether the user is snoring. For example, the control module may compare the signal strength to a (e.g., predetermined) signal strength threshold to determine whether the signal strength exceeds the signal strength threshold. Additionally or alternatively, the control module may be configured to determine whether the signal pattern matches one or more reference signal patterns indicative of snoring. The comparison(s) at S208 may be used to obtain a comparison result that indicates whether or not the user is determined to be snoring. In some embodiments, the reference pattern may indicate a characteristic vibration pattern of snoring, which may be determined based on monitoring the user and/or monitoring a plurality of users.

If the comparison result is No at S208 (e.g., the user is not snoring), method 200 may return to S204, where the snore detection module may be configured to continue to detect snoring, generate the suitable signals, and provide the generated signals to the control module

If the comparison result at S208 is Yes, the user may be determined as snoring and method 200 may proceed to S210. At S210, an actuation module, such as the actuation module 1006, may generate an actuation. The actuation may be configured to provide a stimulation which causes the user to change sleep position with minimal or no impact to the user's sleep. In some embodiments, the stimulation may be applied for a short duration (e.g., a few seconds).

In some embodiments, the actuation module may be configured to use a plurality of predetermined intensity levels that are different from each other. The plurality of intensity levels may include a first, or starting, intensity level, which may be standardized or customized for an individual user (e.g., based on tracking previously effective actuation intensities). In some embodiments, the actuation module may be configured to generate the actual at the first or starting intensity level when the user is first determined to have begun snoring.

At S212 and S214, the control module may be configured to determine whether the user's body has moved. In practice, the actuation at S220 may or may not cause the user to change sleep position or otherwise move in a manner that reduces snoring. In some embodiments, a movement detection module, such as the movement detection module 1008, may detect movement of the user's body. The movement detection module may be further configured to generate a motion signal indicating the movement and/or amount of movement.

At S214, the control module may be configured to determine whether the user's body has moved. If the movement detection module fails to detect a movement (or a sufficient amount of movement), the movement detection module may be configured to generate the motion signal indicating the lack of motion and provide the motion signal to the control module.

Method 200 may proceed to S216, where the control module may be configured to increase the actuation intensity. For example, the lack of user movement may indicate that the actuation intensity is too gentle. Thus the control module may increase the actuation intensity, such to a (e.g., predetermined) next higher intensity level. Method 200 may then return to S212, where the motion movement detection module may be configured to detect body movement, and so forth.

If the movement detection module detects a (e.g., suitable) movement at S214, method 200 may proceed to S218, where the control module may be configured to generate a stop signal and provide the stop signal to the actuation module. In some embodiments, the control module may be further configured to delay for a predetermined period of time before performing an additional actuation. For example, the control module may be configured to delay 30 minutes, and then return to S204, where the snore detection module may be configured to detect vibrations and generate vibration signals, and so forth. The control module may be configured to compare signal strengths and/or signal patterns subsequent to the delay of the predetermined period of time at S208. In general, method 200 may be repeated during the user's sleep to provide continuous snore monitoring and correction.

In some embodiments, method 200 may additionally or alternatively be used for controlling sleep apnea or other sleep-related disorders. For example, in response to determining that the user has sleep apnea, an actuation module, such as the actuation module 1006, may generate an actuation to wake the user. The actuation to wake the user may be at an intensity level that is greater than the one or more intensity levels discussed above that causes the user to change sleep position with minimal or no impact to the user's sleep. Any type of suitable actuation may be used to wake the user, including one or more of a swipe actuator, heat/cold transmission unit, pressure generator and/or an electronic vibration generator. In some embodiments, a feedback technique may also be used for sleep apnea in which user motion or other indicators of being awake can be used to determine whether to increase the actuation intensity.

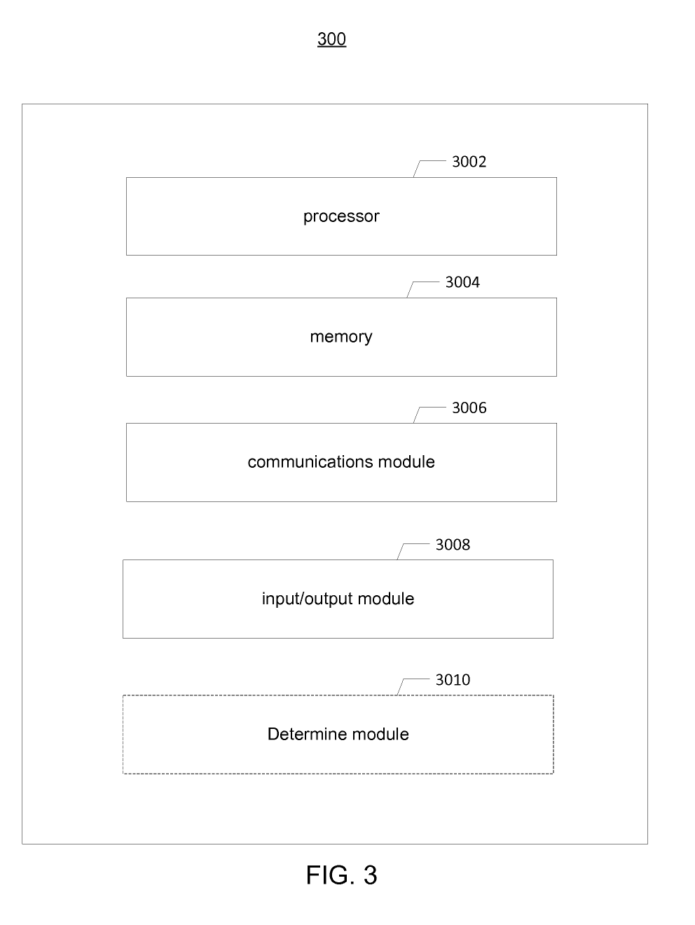

FIG. 3 shows a schematic block diagram of example circuitry 300, some or all of which may be included in detection module 1002, control module 1004, actuation module 1006 and/or movement/position detection module 1008. In accordance with some example embodiments, circuitry 300 may include various elements, such as one or more processors 3002, memories 3004, communications modules 3006, and/or input/output modules 3008.

As referred to herein, “module” includes hardware, software, and/or firmware configured to perform one or more particular functions. In this regard, the means of circuitry as described herein may be embodied as, for example, circuitry, hardware elements (e.g., a suitably programmed processor, combinational logic circuit, integrated circuit, and/or the like), a computer program product comprising computer-readable program instructions stored on a non-transitory computer-readable medium (e.g., memory 3004) that is executable by a suitably configured processing device (e.g., processor 3002), or some combination thereof.

Processor 3002 may, for example, be embodied as various means for processing including one or more microprocessors with accompanying digital signal processor(s), one or more processor(s) without an accompanying digital signal processor, one or more coprocessors, one or more multi-core processors, one or more controllers, processing circuitry, one or more computers, various other processing elements including integrated circuits such as, for example, an ASIC (application specific integrated circuit) or FPGA (field programmable gate array), or some combination thereof. Processor 3002 may comprise a plurality of means for processing. The plurality of means for processing may be embodied on a single computing device or may be distributed across a plurality of computing devices collectively configured to function as circuitry 300. The plurality of means for processing may be in operative communication with each other and may be collectively configured to perform one or more functionalities of circuitry 300 as described herein. In an example embodiment, processor 3002 may be configured to execute instructions stored in memory 3004 or otherwise accessible to processor 3002. These instructions, when executed by processor 3002, may cause circuitry 300 to perform one or more of the functions described herein.

Whether configured by hardware, firmware/software methods, or by a combination thereof, processor 302 may comprise an entity capable of performing operations according to embodiments of the present disclosure while configured accordingly. Thus, for example, when processor 3002 is embodied as an ASIC, FPGA, or the like, processor 3002 may comprise specifically configured hardware for conducting one or more operations described herein. As another example, when processor 3002 may be embodied as an executor of instructions, such as may be stored in memory 3004, the instructions may specifically configure processor 302 to perform one or more algorithms, methods, operations, or functions described herein. For example, processor 3002 may be configured to determine vibration strength and compare the vibration strength to a predetermined vibration strength threshold.

Memory 3004 may comprise, for example, volatile memory, non-volatile memory, or some combination thereof. Although illustrated in FIG. 3 as a single memory, memory 3004 may comprise a plurality of memory components. The plurality of memory components may be embodied on a single computing component or distributed across a plurality of computing components. In various embodiments, memory 3004 may comprise, for example, a hard disk, random access memory, cache memory, flash memory, a compact disc read only memory (CD-ROM), solid state memory, digital versatile disc read only memory (DVD-ROM), an optical disc, circuitry configured to store information, integrated circuitry, chemical/biological memory, paper, or some combination thereof. Memory 3004 may be configured to store information, data, applications, instructions, or the like for enabling circuitry 300 to carry out various functions in accordance with example embodiments discussed herein. For example, in at least some embodiments, memory 3004 may be configured to buffer input data for processing by processor 3002. Additionally or alternatively, in at least some embodiments, memory 3004 may be configured to store program instructions for execution by processor 3002 and/or data for processing by processor 3002. Memory 3004 may store information in the form of static and/or dynamic information. This stored information may be stored and/or used by circuitry 300 during the course of performing its functionalities.

Communications module 3006 may be embodied as any component or means for communication embodied in circuitry, hardware, a computer program product comprising computer readable program instructions stored on a computer readable medium (e.g., memory 3004) and executed by a processing device (e.g., processor 3002), or a combination thereof that is configured to receive and/or transmit data from/to another device, such as, for example, a second circuitry 300 and/or the like. In some embodiments, communications module 3006 (like other components discussed herein) can be at least partially embodied as or otherwise controlled by processor 3002. In this regard, communications module 3006 may be in communication with processor 3002, such as via a bus. Communications module 3006 may include, for example, an antenna, a transmitter, a receiver, a transceiver, network interface card and/or supporting hardware, and/or firmware/software for enabling communications. Communications module 3006 may be configured to receive and/or transmit any data that may be stored by memory 3004 using any protocol that may be used for communications. Communications module 3006 may additionally and/or alternatively be in communication with the memory 3004, input/output module 3008, and/or any other component of circuitry 300, such as via a bus. Communications module 3006 may be configured to use one or more communications protocols such as, for example, short messaging service (SMS), Wi-Fi (e.g., a 802.11 protocol, Bluetooth, etc.), radio frequency systems (e.g., 900 MHz, 1.4 GHz, and 5.6 GHz communication systems), infrared, GSM, GSM plus EDGE, CDMA, quadband, and other cellular protocols, VOIP, or any other suitable protocol.

Input/output module 3008 may be in communication with processor 3002 to receive an indication of an input and/or to provide an audible, visual, mechanical, or other output. In that sense, input/output module 3008 may include means for implementing analog-to-digital and/or digital-to-analog data conversions. Input/output module 3008 may include support, for example, for a display, touch screen, keyboard, button, joystick, mouse, click wheel, an image capturing device, microphone, speaker, biometric scanner, and/or other input/output mechanisms. In embodiments where circuitry 300 may be implemented as a server or database, aspects of input/output module 3008 may be reduced as compared to embodiments where circuitry 300 may be implemented as an end-user machine or other type of device designed for complex user interactions. In some embodiments wherein circuitry 300 is embodied as a server or database, at least some aspects of input/output module 3008 may be embodied on an apparatus used by a user that is in communication with circuitry 300. Input/output module 3008 may be in communication with memory 3004, communications module 3006, and/or any other component(s), such as via a bus. Although more than one input/output module and/or other component can be included in circuitry 300, only one is shown in FIG. 3 to avoid overcomplicating the disclosure (e.g., like the other components discussed herein).

In some embodiments, determining module 3010 may also or instead be included and configured to perform the functionality discussed herein related to determining the pre-defined control operation. In some embodiments, some or all of the functionality of determining module 3010 may be performed by processor 3002. In this regard, the example processes discussed herein can be performed by at least one processor 3002 and/or determining module 3010. For example, non-transitory computer readable storage media can be configured to store firmware, one or more application programs, and/or other software, which include instructions and other computer-readable program code portions that can be executed to control processors of the components of circuitry 300 to implement various operations, including the examples shown herein. As such, a series of computer-readable program code portions may be embodied in one or more computer program products and can be used, with a device, server, database, and/or other programmable apparatus, to produce the machine-implemented processes discussed herein.

Any such computer program instructions and/or other type of code may be loaded onto a computer, processor, and/or other programmable apparatus's circuitry to produce a machine, such that the computer, processor, or other programmable circuitry that executes the code may be the means for implementing various functions, including those described herein. In some embodiments, one or more external systems (such as a remote cloud computing and/or data storage system) may also be leveraged to provide at least some of the functionality discussed herein.

As described above and as will be appreciated based on this disclosure, various embodiments may be implemented as methods, mediums, devices, servers, databases, systems, and the like. Accordingly, embodiments may comprise various forms, including entirely of hardware or any combination of software and hardware. Furthermore, embodiments may take the form of a computer program product on at least one non-transitory computer readable storage medium having computer readable program instructions (e.g., computer software) embodied in the storage medium. Any suitable computer-readable storage medium may be utilized including non-transitory hard disks, CD/DVD-ROMs, flash memory, optical storage devices, quantum storage devices, chemical storage devices, biological storage devices, magnetic storage devices, etc.

Embodiments have been described above with reference to components, such as functional modules, system components, and circuitry. Also described are example process flow charts describing functionality that may be implemented by one or more components and/or means discussed above and/or other suitably configured circuitry.

According to one aspect of the subject disclosure, one or more components discussed herein may operate under control of a computer program. The computer program for performing the methods of exemplary embodiments of the disclosure may include one or more computer-readable program code portions, such as a series of computer instructions, embodied or otherwise stored in a computer-readable storage medium, such as the non-volatile storage medium.

It will be understood that each block or step of the flow chart (e.g., as shown in FIG. 2), and combinations of blocks or steps in the flow chart, may be implemented by various means, such as hardware alone or in combination with firmware, and/or software including one or more computer program instructions. As will be appreciated, any such computer program instructions may be loaded onto a computer, special purpose computer, a smart phone, or other programmable data processing apparatus, such as processor 3002, to produce a machine, or machines, such that the computer program product includes the instructions which execute on the computer or other programmable data processing apparatus (e.g., hardware) to create means for implementing the functions described herein, such as the functions specified in the block(s) or step(s) of the flow chart of FIG. 2.

These computer program instructions may also be stored in a computer-readable storage device (e.g., memory 3004) that may direct a computer or other programmable data processing apparatus to function in a particular manner, such that the instructions stored in the computer-readable storage device produce an article of manufacture including instruction computer-readable instructions for implementing the functions described herein, such as the functions specified in the block(s) or step(s) of the flow chart of FIG. 2. The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions described herein, such as the functions specified in the block(s) or step(s) of the flow chart of FIG. 2.

Accordingly, blocks or steps of the flow chart support means and combinations of means for performing and/or implementing the specified functions, combinations of steps for performing and/or implementing the specified functions and program instruction means for performing and/or implementing the specified functions. It will also be understood that one or more blocks or steps of the flow chart, and combinations of blocks or steps in the flow chart, may be implemented by special purpose hardware-based computer systems which perform the specified functions or steps, or combinations of special purpose hardware and computer instructions.

It will be appreciated by those skilled in the art that changes could be made to the examples described above without departing from the broad inventive concept. It is understood, therefore, that this disclosure is not limited to the particular examples disclosed, but it is intended to cover modifications within the spirit and scope of the disclosure as defined by the appended claims. Although specific terms are employed herein, they are used in a generic and descriptive sense only and not for purposes of limitation.

Although various embodiments have been described with reference to the figures, those skilled in the art should understand that various modification may also be occur to various embodiments without departing from the scope of this disclosure. Therefore, the scope of protection of the present invention should be determined by the contents of the appended claims.

Those skilled in the art should understand that each module or each step of the present invention may be implemented by a general purpose computing device, may be focused on a single computing device, or may be distributed on the network composed of multiple computing devices. Optionally, they may be implemented by a computing device executable program code, so that they are stored in a storage device for execution by the computing device, or may be implemented by making them into various integrated circuit module respectively, or making multiple modules or steps among them into a single integrated circuit module. In this way, various embodiments will not limit combinations of any specific hardware and software

Various alterations or changes will be apparent to one of skill in the art having the benefit of the embodiments herein. Any modification, equivalent replacement, improvement, etc. within the spirit and principle of the disclosure is be contained within the scope of the discussion herein.

1. A sleep control system, comprising:
a snore detection module configured to:
detect vibrations caused by snoring of a user; and
generate vibration signals indicating the vibrations;
a control module configured to:
determine a vibration strength based upon the vibration signals;
determine a vibration strength threshold; and
determine whether the vibration strength exceeds the vibration strength threshold; and
in response to determining that vibration strength exceeds the vibration strength threshold, provide an actuation signal to an actuation module; and
the actuation module configured to generate an actuation to stimulate movement of the user in response to receiving the actuation signal.

2. The system of claim 1, wherein the control module is further configured to:
determine a vibration pattern based on the vibration signals;
determine a reference vibration pattern representative of snoring of the user;
determine whether the vibration pattern corresponds with the reference vibration pattern; and
in response to determining that the vibration pattern corresponds with the reference vibration pattern, provide the actuation signal to an actuation module.

3. The system of claim 1 further comprises a movement detection module configured to detect a movement of the user and generate a motion signal indicating the movement.

4. The system of claim 3, wherein:
the control module is further configured to:
receive the motion signal from the movement detection module;
determine, based on the motion signal, whether the user has moved; and
in response to determining that the user failed to move, provide a second actuation signal to the actuation module to increase an intensity of the actuation; and
the actuation module is further configured to increase the intensity of the actuation based on receiving the second actuation signal.

5. The system of claim 3, wherein:
the control module is further configured to:
receive the motion signal from the movement detection module;
determine, based on the motion signal, whether the user has moved; and
in response to determining that the user has moved, provide a stop signal to the actuation module to stop the actuation; and
the actuation module is further configured to stop the actuation based on receiving the stop signal.

6. The system of claim 3, wherein the control module is further configured to:
receive the motion signal from the movement detection module;
determine, based on the motion signal, whether the user has moved; and
in response to determining that the user has moved, determine at least one of a second vibration strength and a second vibration pattern at a predetermined amount of time subsequent to the user movement.

7. The system of claim 1, wherein the snore detection module and the actuation module are included with a wearable device that is separate from a processing device including the control module.

8. The system of claim 1, wherein the actuation module is further configured to increase an intensity of the actuation based on changing one or more of a frequency, strength, amplitude, temperature, pressure, and air flow of the actuation.

9. The system of claim 1, wherein the snore detection module comprises a vibration detector including an ionic polymer metal composite (IPMC) including an ion exchange membrane and an electro-less plating of metal, the metal comprising one or more of nickel, copper, silver, gold, platinum, and palladium.

10. The system of claim 1, wherein the actuation comprises at least one of a massage stimulation, pressure stimulation, vibration stimulation, air blow stimulation, heat stimulation, cold stimulation and electrical stimulation.

11. The system of claim 1, wherein:
the control module is further configured to:
determine the user has sleep apnea based at least in part on the vibration signals; and
in response to determining that the user has sleep apnea, provide a second actuation signal to the actuation module; and
the actuation module is further configured to generate a second actuation to wake the user in response to receiving the second actuation signal, the second actuation including a higher intensity level than the actuation to stimulate movement of the user.

12. The system of claim 11, wherein:
the snore detection module further includes at least one sleep sensor configured to generate sensor data, the at least one sensor including at least one of a microphone detector, an eye movement sensor, an electrocardiogram (ECG) sensor, a blood oxygen sensor, or a brain wave sensors; and
the control module is further configured to determine the user has sleep apnea based at least in part on the vibration signals and sensor data.

13. A machine-implemented method of sleep control, comprising:
detecting, by a snore detection module, vibrations caused by snoring of a user;
generating, by the snore detection module, vibration signals indicating the vibrations;
determining, by a control module, a vibration strength based upon the vibration signals;
determining, by the control module, a vibration strength threshold;
determining, by the control module, whether the vibration strength exceeds the vibration strength threshold;
in response to determining that vibration strength exceeds the vibration strength threshold, providing, by the control module, an actuation signal to an actuation module; and
generating, by an actuation module, an actuation to stimulate movement of the user in response to receiving the actuation signal.

14. The method of claim 13 further comprising, by the control module:
determining a vibration pattern based on the vibration signals;
determining a reference vibration pattern representative of snoring of the user;
determining whether the vibration pattern corresponds with the reference vibration pattern; and
in response to determining that the vibration pattern corresponds with the reference vibration pattern, providing the actuation signal to an actuation module.

15. The method of claim 13 further comprising, by a movement detection module:
detecting a movement of the user; and
generating a motion signal indicating the movement.

16. The method of claim 15 further comprising:
receiving, by the control module, the motion signal from the movement detection module;
determining, by the control module and based on the motion signal, whether the user has moved;
in response to determining that the user failed to move, providing, by the control module, a second actuation signal to the actuation module to increase intensity of the actuation; and
increasing, by the actuation module, the intensity of the actuation based on receiving the second actuation signal.

17. The method of claim 15 further comprising:
receiving, by the control module, the motion signal from the movement detection module;
determining, by the control module and based on the motion signal, whether the user has moved;
in response to determining that the user has moved, provide a stop signal to the actuation module to stop the actuation; and
stopping, by the actuation module, the actuation based on receiving the stop signal.

18. The method of claim 15 further comprising, by the control module:
receiving the motion signal from the movement detection module;
determining, based on the motion signal, whether the user has moved;
in response to determining that the user has moved, determining at least one of a second vibration strength and a second vibration pattern at a predetermined amount of time subsequent to the user movement.

19. The method of claim 13, wherein the snore detection module and the actuation module are included with a wearable device that is separate from a processing device including the control module.

20. The method of claim 13 further comprising increasing, by the actuation module, an intensity of the actuation based on changing one or more of a frequency, strength, amplitude, temperature, pressure, and air flow of the actuation.

21. The method of claim 13, wherein the snore detection module comprises a vibration detector including an ionic polymer metal composite (IPMC) including an ion exchange membrane and an electro-less plating of metal, the metal comprising one or more of nickel, copper, silver, gold, platinum, and palladium.

22. The method of claim 13, wherein the actuation comprises at least one of a massage stimulation, pressure stimulation, vibration stimulation, air blow stimulation, heat stimulation, cold stimulation and electrical stimulation.

23. The method of claim 13 further comprising:
determining, by the control module, the user has sleep apnea based at least in part on the vibration signals;
in response to determining that the user has sleep apnea, providing, by the control module, a second actuation signal to the actuation module; and
generating, by the actuation module, a second actuation to wake the user in response to receiving the second actuation signal, the second actuation including a higher intensity level than the actuation to stimulate movement of the user

24. The method of claim 23 further comprising:
generating sensor data, by a sleep sensor, wherein the sleep sensor includes at least one of a microphone detector, an eye movement sensor, an electrocardiogram (ECG) sensor, a blood oxygen sensor, or a brain wave sensors; and
determining, by the control module, the user has sleep apnea based at least in part on the vibration signals and sensor data.

25-31. (canceled)

32. An apparatus, comprising:
circuitry configured to:
receive vibration signals from a snore detection module indicating vibrations caused by snoring;
determine a vibration strength based upon the vibration signals;
determine a vibration strength threshold;
determine a vibration pattern based on the vibration signals determine a reference vibration pattern representative of snoring of the user;
determine whether the vibration strength exceeds the vibration strength threshold and whether the vibration pattern corresponds with the reference vibration pattern; and
in response to determining that vibration strength exceeds the vibration strength threshold and the vibration pattern corresponds with the reference vibration pattern, provide an actuation signal to an actuation module.

33-41. (canceled)
